(19) United States
(12) Patent Application Publication
(10) Pub. No.: US 2006/0115426 A1
(43) Pub. Date: Jun. 1, 2006

(54) Methods of detecting breast cancer, brain cancer, and pancreatic cancer

(76) Inventors: Jamey P. Weichert, Fitchburg, WI (US); Marc A. Longino, Verona, WI (US); Amy R. Moser, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/201,793

(22) Filed: Aug. 11, 2005

Related U.S. Application Data
(60) Provisional application No. 60/600,588, filed on Aug. 11, 2004.

(51) Int. Cl. A61K 51/00; C07F 9/02

(52) U.S. Cl. 424/1.11; 554/78

(57) ABSTRACT

The present invention discloses methods for detecting breast cancer, brain cancer, and pancreatic cancer via nuclear imaging using certain phospholipid ether analogs.

### CROSS-REFERENCE TO RELATED APPLICATIONS

This application claims the benefit of U.S. provisional patent application Ser. No. 60/600,588, filed on Aug. 11, 2004, incorporated by reference in its entirety.

### STATEMENT REGARDING FEDERALLY SPONSORED RESEARCH OR DEVELOPMENT

This invention was made with United States government support awarded by the following agencies: ARMY/MRMC DAMD17-02-1-0627 and NIH CA095249. The United States has certain rights in this invention.

### BACKGROUND OF THE INVENTION

The early detection of cancer has been one of the primary goals of modem imaging technology, since the identification of a suspected tumor in a localized stage significantly improves the chances for successful treatment and elimination of the cancerous tissue. A large number of imaging strategies have therefore been designed, using a variety of techniques and modalities, to aid the physician in making an accurate diagnosis as early as possible.

Unfortunately, conventional imaging techniques such as computerized tomography (CT) and magnetic resonance imaging (MRI) are limited in their ability to afford a conclusive diagnosis of a suspected lesion, since they are only capable of observing differences in the density or morphology of tissues. A more invasive and costly biopsy procedure is often necessary to provide a definitive diagnosis. In contrast, nuclear medicine techniques such as positron emission tomography (PET) and single photon emission tomography (SPECT) can provide functional or biochemical information about a particular organ or area of interest.

U.S. Pat. Nos. 6,255,519 and 6,417,384 (both are herein incorporated by reference in their entirety) disclosed a series of radioiodinated phospholipid ether analogs with significant tumor avidity that can be used along with PET and SPECT for imaging and visualization of tumors. Although the precise mechanism of action is not fully understood, the prevailing hypothesis is that while normal cells can metabolize and clear these phospholipid ether analogs, tumor cells cannot and this leads to selective entrapment of the analogs in tumor cell membranes.

Although the suitability of using the above phospholipid ether analogs as tumor imaging agents have been demonstrated in general in a variety of rodent and animal tumor models (see e.g., Rampy M A, et al. J. Nucl. Med. 37:1540-1545, 1996), certain imaging applications may have unique requirements that make the feasibility of using the above phospholipid ether analogs unclear. In the case of breast cancer, for example, it is not clear whether the above phospholipid ether analogs can distinguish cancerous tissue from hyperplastic tissue. In the case of brain cancer, it is not clear whether the phospholipid ether analogs can cross the blood-brain barrier and the effect thereof on distinguishing cancerous tissue from normal tissue in the brain. Imaging of pancreatic cancer is typically difficult due to interfering signals from inflammatory regions and other factors.

### BRIEF SUMMARY OF THE INVENTION

Using 18-(p-iodophenyl)-octadecylphosphocholine (referred to as NM404) as an example, the inventors have discovered that phospholipid ether analogs defined by formula I and II provided in the “detailed description of the invention” can be used to distinguish between malignant breast/mammary tumors and hyperplastic breast/mammary tissues as these analogs selectively accumulate in the malignant breast/mammary tumor cells versus the hyperplastic breast/mammary cells. The inventors found that the accumulation in the hyperplastic breast/mammary cells is similar to that of normal breast/mammary cells. It is expected that the accumulation in benign breast/mammary tumor cells will be similar to that in hyperplastic cells. Therefore, these analogs can also be used to distinguish between malignant and benign breast/mammary tumors.

Also using NM404 as an example, the inventors have discovered that the above phospholipid ether analogs, when injected intravenously, can reach and then selectively accumulate in brain tumors versus surrounding normal brain regions. Therefore, these analogs can be used as imaging agents for brain tumors. In addition, clear pancreatic cancer imaging can be achieved with these analogs as well.

The disclosure here provides new tools for diagnosing breast/mammary cancer, brain cancer, and pancreatic cancer, for determining the extent and location of post-operative residual tumor in the above cancer types, for surveillance after primary tumor resection in the above cancer types, and for determining the response of the above cancer types to chemotherapy. In the case of breast cancer diagnosis, the use of the above phospholipid ether analogs can reduce or eliminate false positives from hyperplastic and benign tumor tissues. In particular for brain tumors, these analogs are useful for differentiating brain edema surrounding a brain tumor from small additional foci of tumor. This has obvious implications for surgical approaches to these tumors as well as targeting of radiotherapy, particularly in light of advances in conformal delivery and tomotherapy.

### BRIEF DESCRIPTION OF THE SEVERAL VIEWS OF THE DRAWINGS

FIG. 1 shows NM404 Bioscan images of Min mouse with spontaneous right axillary mammary adenocarcinoma (10 mm in diameter) at various times following intravenous administration of ¹²⁵I-NM404 (15 μCi). Coronal microCT image (non-contrast-enhanced) is shown for anatomic comparison (left panel, T=tumor).

FIG. 2 shows Bioscan image of excised mammary glands (A) and colon (E) from an FVBxB6 Min mouse 8 days post NM404 administration. Corresponding digital photo of same excised tissues are shown in B and D, respectively. Carmine stained enlarged photograph (C) shows the presence of hyperplasias (arrows) but no corresponding focal activity in the Bioscan Image (A). Tumor uptake on Bioscan image (A) corresponds to larger adenocarcinoma in B. Photograph (D) and Bioscan image (E) of excised colon indicates no uptake of NM404 in adenomatous polyps (arrows).

FIG. 3 shows MicroCT scans of same Min mouse in FIG. 2. Panel A is a low density surface rendering showing a large left axial mammary tumor. Panel B is the high density surface rendering after blood pool CT contrast agent BP10 was administered to help locate tumor feeder vessels. Panel C is a composite coronal CT image and high density surface rendering showing absolute feeder vessel localization. Orientation is from beneath in panel C, whereas Panels A and B are viewed from above.

FIG. 4 shows digital photograph (A) and corresponding Bioscan image of excised C6-glioma bearing rat brain (B) 4 days after intravenous injection of ¹²⁵I-NM404. Position and size-matched fused Bioscan image and photograph (C) indicates intense localization of NM404 in tumor. The presence of tumor was histologically confirmed in H&E stained sample in D. Screening axial MRI scan (E) obtained several days prior to NM404 injection initially confirmed the presence of a glioma.

FIG. 5 shows fused 3D surface-rendered MRI image and 3D microPET image (A) obtained 24 h after i.v. injection of 1²⁴I-NM404 (100 μCi) into a rat with a CNS-1 glioma brain tumor. Images were fused using Amira (v3.1). Right panels show (B) contrast-enhanced coronal MRI slice through the tumor (arrow) and (C) fused coronal MRI and ¹²⁴I-NM404 microPET images corroborating presence and location of the tumor.

FIG. 6 shows Bioscan images of c-myc pancreatic tumor mouse 4-days post ¹²⁵I-NM404 administration. In vivo image (A) compared with digital photo of dissected mouse (B) showing presence of a large (2 cm) pancreatic tumor (T). Three tumors were excised and the remaining carcass scanned (C). The excised tumors were scanned (D) for comparison with digital photo (E). Color scale ranges from 0 (black) to 40 (white) cpm.

FIG. 7 shows the fused in vivo Bioscan/digital photo image of c-myc pancreatic tumor mouse 4 days post ¹²⁵I-NM404 injection (A) and ex vivo image of excised tumors (B) for comparison with digital photo (C). Color range is the same as in FIG. 6.

FIG. 8 shows MicroCT axial scans of pancreatic tumor-bearing mice. Two large tumors (T) are easily seen in the axial image in panel A. Image of a different mouse in B depicts a pancreatic tumor (arrow) located adjacent to the spleen. In mice, the pancreas is a ubiquitous tissue. A digital photo of the excised spleen and attached tumor is shown in panel C for comparison.

### DETAILED DESCRIPTION OF THE INVENTION

The phospholipid ether analogs that can be used for imaging malignant breast/mammary tumors, brain tumors, and pancreatic tumors are defined by formula I and II: 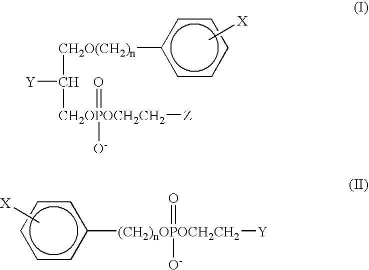
wherein in formula I X is a radioactive isotope of a halogen, n is an integer between 16 and 30, Y is selected from the group consisting of H, OH, COOH, O(C═O)R, and OR, and Z is selected from the group consisting of NH₂, NR₂, and NR₃, wherein R is an alkyl or aralkyl substituent; and wherein in formula II X is a radioactive isotope of a halogen, n is an integer between 16 and 30, and Y is selected from the group comprising NH₂, NR₂, and NR₃, wherein R is an alkyl or aralkyl substituent.

It is well with the capability of a skilled artisan to make the above phospholipid ether analogs and further prepare them for administering to a human or non-human animal for tumor imaging. For example, the phospholipid ether analogs can be labeled with iodine radioisotopes using an isotope exchange method as described in Weichert J P, et al. (Int J Appl Rad Isotopes 37:907-913, 1986, incorporated herein by reference in its entirety) and prepared for injection as described in Rampy M A, et al. (J Nucl Med. 37:1540-1545, 1996, incorporated herein by reference in its entirety). U.S. Pat. Nos. 6,255,519 and 6,417,384 also provide methods for making the phospholipid ether analogs as well as particular embodiments of the analogs. Specific examples for preparing ¹²⁴I-NM404 for clinical use and for performing ¹²⁴I-NM404-PET imaging in patients are provided below.

In one embodiment of the analogs defined by formula I, n is 18, Y is hydrogen, and Z is N(CH₃)₃. In one embodiment of the analogs defined by formula II, n is 18 and Y is N(CH₃)₃ (NM404).

Radioactive iodine isotopes such as ¹²²I, ¹²³I, ¹²⁴I, ¹²⁵I, and ¹³¹I are preferred isotopes for labeling the phospholipid ether analogs. Among them, ¹²⁴I is the most preferred isotope. ¹²⁴I has been used for PET imaging in various animal and experimental models (see e.g., Frey P, et al. Eur J Nucl Med 10:472-476, 1985; Ott R J, et al. Br J Rad 60:245-251, 1987; Langen K J, et al. J Nucl Med 31:281-286, 1990; Snook D E, et al. Br J Cancer 62:89-91, 1990; Wilson C B, et al. Int J Cancer 47:344-347, 1991; Westera G, et al. Nucl Med Comm 12:429-437, 1991; Larson S M, et al. J Nucl Med 33:2020-2023, 1992; Bakir M A, et al. J Nucl Med, 33:2154-2160, 1992 (Erratum in: J Nucl Med 34:290, 1993); Sundaresan G, et al. J Nucl Med 44:1962-1969, 2003; and Lee F T, et al. J Nucl Med 42:764-769, 2001) and it has also shown dosimetric and imaging characteristics suitable for human use (Jacobs A, et al. J Nucl. Med. 42:467-475, 2001). ¹²⁴I is a positron emitting radionuclide with a half-life of 4.2 days and this matches well with phospholipid ether tumor uptake and retention kinetics (Pentlow K S, et al. J Nucl Med 37:1557-1562, 1996). One study showed that PET imaging with ¹²⁴I affords a higher sensitivity than planer ¹³¹I-gamma imaging (Blasberg R G, et al. Cancer Research 60:624-635, 2000). In comparison to traditional gamma camera imaging, PET also offers significant resolution enhancement and 3-dimensional capabilities.

In one aspect, the present invention relates to a method for detecting malignant breast/mammary tumor but not hyperplasia or benign tumor in a breast/mammary tissue of a human or non-human animal. The method involves administering a phospholipid ether analog described above to the human or non-human animal and determining whether one breast/mammary region of the human or non-human animal retains a higher level of the analog than surrounding breast/mammary regions wherein a higher retention region indicates the location of malignant breast/mammary tumor but not hyperplasia or benign tumor.

In another aspect, the present invention relates to a method for detecting brain tumor in a human or non-human animal that has or is suspected of having a brain tumor. The method involves administering a phospholipid ether analog described above to the human or non-human animal and determining whether one brain region of the human or non-human animal retains a higher level of the analog than surrounding brain region wherein the presence of a higher retention region indicates that the human or non-human animal has a brain tumor. In one embodiment, the brain tumor being detected is glioma or astrocytoma.

In yet another aspect, the present invention relates to a method for detecting pancreatic cancer in a human or non-human animal that has or is suspected of having pancreatic cancer. The method involves administering a phospholipid ether analog described above to the human or non-human animal and determining whether one pancreatic region of the human or non-human animal retains a higher level of the analog than surrounding pancreatic region(s) wherein a higher retention region indicates that the human or non-human animal has pancreatic cancer.

While intravenous injection is the preferred method of administering a phospholipid ether analog for the purpose of the prevent invention, other suitable systemic and topical routes can also be utilized.

The invention will be more fully understood upon consideration of the following non-limiting examples.

### EXAMPLE 1

### Specificity of NM404 for Neoplasia versus Hyperplasia in the Apc^{Min/+} Endogenous Mammary Adenocarcinoma Model

Materials and Methods

Apc^{Min/+} Mouse Model: This model is comprised of mice carrying the Min allele of Apc (Apc^{Min/+} mice) and the type of lesions that appear in these mice are molecularly and histologically similar to breast cancer in humans. This model offers specific advantages over xenograft models in that female Apc^{Min/+} mice are predisposed to developing mammary hyperplasias and carcinomas and intestinal adenomas. On the C57BL6/J genetic background, about 5% of untreated females will develop a mammary tumor by 100 days of age (Moser A R, et al. Proc. Natl. Acad. Sci. USA 90:8977-81, 1993). The incidence and multiplicity of the mammary lesions can be increased by a single dose of ethylnitrosourea (ENU), a direct acting alkylating agent. Treatment with ENU results in 90% of B6 Apc^{Min/+} females developing an average of 3 mammary squamous cell carcinomas (SCC), but few hyperplasic lesions within 60 days after treatment.

Genetic background can affect the incidence, latency, and type of mammary lesions that develop. For example, FVBxB6 Apc^{Min/+} female mice develop an average of 0.2 mammary tumors per mouse, but 4 hyperplasias per mouse within 120 days of treatment. BALB/xB6 Apc^{Min/+} develop an average of 1.8 mammary tumors and 0.6 hyperplasias per mouse (Moser A R, et al. Cancer Research 61:3480-3485, 2001). FVBxB6 and BALBxB6 Apc^{Min/+} mice develop both mammary SCC and adenocarcinomas (AC).

Imaging Studies: NM404 (100 μg) was radioiodinated with ¹²⁵I via isotope exchange in a melt of privalic acid. Following HPLC purification it was dissolved in an aqueous 2% tween-20 solution prior to tail vein injection (15 μCi/20 g mouse) into 6 female Apc^{Min/+} mice. Mice were anesthetized and scanned for up to 30 days post injection on a modified Bioscan AR2000 radio-TLC scanner (1 mm increments at 2 mm acquisition/lane and 1 mm high-resolution collimator) and also in an ImTek microCT scanner (390 steps) for anatomic comparison. MicroCT images were displayed using Amira software. At sacrifice, mammary glands or excised tumors were imaged ex vivo, lesions were excised, weighed, and radioactivity quantitated. Lesion samples were submitted for histological classification. If necessary a long-acting CT blood pool contrast agent (BP20), as described in Weichert J P, et al. Radiology 216:865-871, 2000 and suitable for long microCT acquisitions times, was injected intravenously prior to CT scanning in order to assist in blood vessel visualization (FIG. 3).

Results

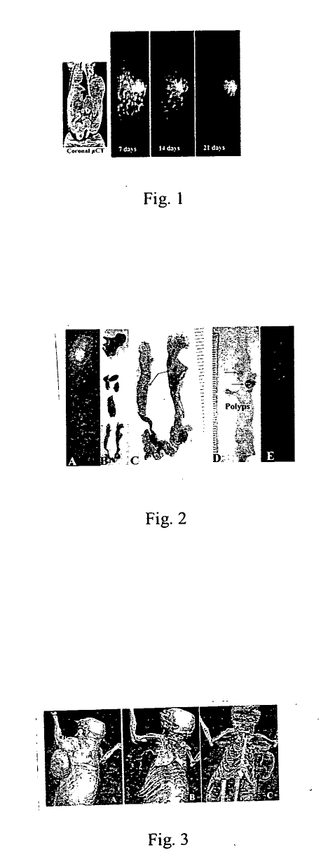

This model is unique in that hyperplastic mammary lesions, mammary carcinomas, and intestinal adenomas develop in the same mouse. Imaging results with NM404 (FIGS. 1 and 2) have shown striking uptake (>20% dose/g) and prolonged retention in all spontaneous mammary carcinomas ranging from 2-15 mm in diameter. Although tumor localization appears rapid, background radioactivity may persist for several days in liver and gut during the body clearance phase. HPLC analysis of radioactive urine and feces indicated the presence of metabolites and no parent NM404. Tumor retention of NM404 persisted for >21 days, the predetermined study endpoint. NM404 did not localize, however, in either focal alveolar hyperplasias or in intestinal adenomatous polyps found frequently in these mice (FIG. 2).

MicroCT images confirmed the presence and precise location of all mammary tumors (FIG. 3). NM404 apparently is metabolized and cleared from normal cells but becomes metabolically trapped in malignant tumor cell membranes.

### EXAMPLE 2

### Imaging of Intracranial Gliomas Using Radioiodinated NM404

Materials and Methods

Glioma tumor model: All animals were housed and handled in accordance with the University of Wisconsin Research Animal Resources Center guidelines. Rat C6 glioma cells were propagated in DMEM medium (Life Technologies, Gaithersburg, Md.) supplemented with 10% heat-inactivated FBS (BioWhittaker, Walkersville, MD), 100 U/ml penicillin-G, 100 μg/ml streptomycin, and 0.01 M hepes (Life Technologies, Gaithersburg, Md.). Intracranial tumor implantation was performed as described in Badie B, et al. J. Neuroimmunol. 133:39-45, 2002. Briefly, 1×10⁶ C6 cells were resuspended in 5 μl of 1.2% methylcellulose and injected into the frontal lobes of anesthetized female Wistar rats (Harlan, Indianapolis, Ind.). Sham-operated animals received intracranial injections of an equal volume of methylcellulose without tumor cells.

Imaging studies: Ten days after implantation rats were screened for the presence of intracranial tumors with MRI as described in Badie B, et al. Clin. Cancer Res. 9:872-877, 2003. Briefly, anesthetized rats received 2 ml of Gadodiamide (Gd, Omniscan 287 mg/ml, Nycomed, Princeton, N.J.) intraperitoneally and imaged 10 min later using a 1.5 Tesla clinical MR system (GE Signa LX) and a GE phased array extremity coil. The T1-weighted (TR=500 ms, TE=16.5 ms) multislice sequences covering the entire brain of each rat were inspected to select tumor-bearing rats with varying tumor sizes, and sham-operated rats for NM404 injections.

NM404 (100 μg) was radioiodinated with ¹²⁵I via isotope exchange in a melt of pivalic acid (Weichert J P, et al. Int J Appl Rad Isotopes 37:907-913,1986). Following HPLC purification, NM404 was dissolved in an aqueous 2% tween-20 solution and filtered (0.2 micron filter) prior to tail vein injection (15 μCi/200 g rat) into four tumor-bearing and three sham-operated animals. Animals were euthanized at 24 (n=1), 48 (n=1), and 96 hours (n=2) after NM404 injection, brains were harvested, imaged on a modified Bioscan AR2000 radio-TLC scanner (Bioscan, Inc., Washington, D.C., 1 mm lane increments at 2 min acquisition/lane and using a 1 mm high-resolution collimator), and fixed in 10% buffered formalin. Excised brains were positioned and digitally photographed prior to scanning. Co-registered Bioscan images and digital photographs were fused (Corel Photo-Paint) in order to accurately determine the location of radioactivity. Following imaging, brain sections were prepared by paraffin embedment, sectioning and staining with H&E for histological tumor confirmation.

Biodistribution studies: Rats bearing C6 gliomas (n=4, 10 day old tumors), and sham-operated rats (n=3) were injected with ¹²⁵I-NM404 (15 μCi/200 g rat) and biodistribution studies performed as described in Rampy M A, et al. J. Med. Chem. 38:3156-3162, 1995. Accordingly, animals were euthanized by exsanguination at 24, 48, and 96 hours following injection. A total of 17 tissues including blood, plasma, adrenal glands, bladder wall, bone marrow, brain, eye lens, fat, heart, kidney, liver, lung, muscle, spleen, skin, thyroid, and tumor were excised, rinsed, and dissected free of extraneous tissue. Tissues were minced and duplicate samples weighed and placed in plastic tubes for isotope counting in a gamma counter (Perkin Elmer/Wallac Wizard-1470). Injection site and residual carcass radioactivity were determined in a well counter (Capintec CRC-15R). Time normalized tissue distribution tables were generated by a custom computer program which produces decay-corrected tissue radioactivity concentration data on a percent injected dose/g, % kg dose, and percent injected dose/organ±SEM basis.

Results

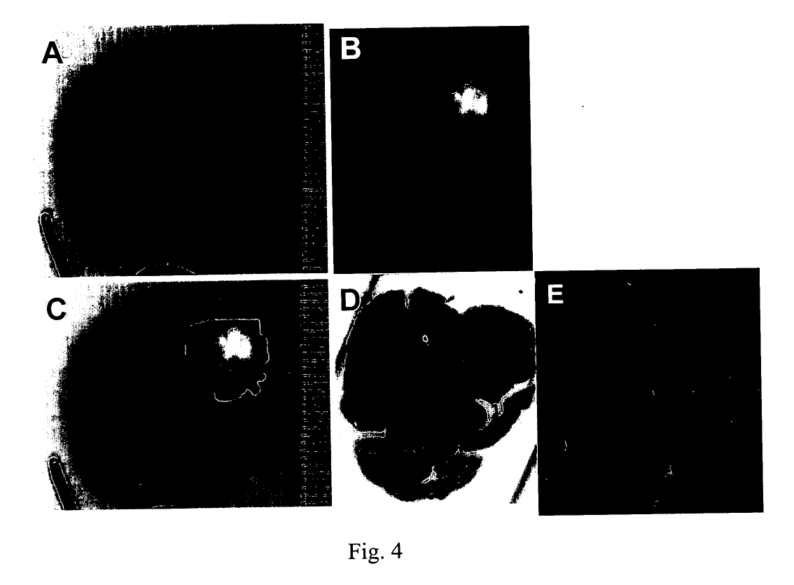

Imaging study: All animals survived to complete the study without complications. By gross pathology, gliomas measured 3-5 mm in diameter. In all tumored animals, gross tracer uptake in C6 gliomas was seen on Bioscan images at all time points. Radioactivity in normal brain tissue was minimal in sham operated control animals, whereas NM404 concentrated intensely in gliomas (FIG. 4). The radiologic-pathologic correlation using Bioscan images superimposed on the gross brain slices demonstrated a precise localization of activity in tumor. Therefore, NM404 is a powerful for visualizing brain tumors including small invasive tumor loci.

Tissue distribution: Tumor-to-brain ratios (based on % injected dose/g, Table 1) in C6-bearing rats were 10.6, 12.0, and 7.8 at 24, 48, and 96 h, respectively. Thyroid radioactivity levels remained low (2.3 and 9.0% injected dose/g at 48 and 96 h, respectively) indicating the stability of the agent with respect to in vivo deiodination.

TABLE 1
Tissue distribution of ¹²⁵I-NM404 in glioma-bearing rats.

| | Time | | |
|---|---|---|---|
| Tissue | 24 h | 48 h | 96 h |
| Blood | 1.83 | 1.16 | 0.82 ± 0.01 |
| Brain | 0.07 | 0.05 | 0.09 ± 0.02 |
| Kidney | 0.91 | 0.44 | 0.45 ± 0.05 |
| Liver | 0.77 | 0.30 | 0.39 ± 0.01 |
| Spleen | 0.80 | 0.33 | 0.33 ± 0.03 |
| Thyroid | NA | 2.34 | 9.06 ± 3.84 |
| Glioma | 0.74 | 0.60 | 0.70 ± 0.04 |
| Tumor/brain | 10.6 | 12.0 | 7.8 |

Results presented as % injected dose/g tissue ± SEM where appropriate.
N = 1 for 24 and 48 h and N = 2 for 96 h.

### EXAMPLE 3

MicroPET Evaluation of ¹²⁴I labeled NM404 in a Rat CNS-1 Brain Tumor Model

Materials and Methods

NM404 was radiolabeled with ¹²⁴I in excellent radiochemical yield (>60% isolated yield, >99% purity) via isotope exchange reaction of stable NM404 with sodium-iodide (Eastern Isotopes, Sterling Va.). Following preparative HPLC purification ¹²⁴I-NM404 was solubilized in 2% Tween-20 and filtered (0.22 micron). NM404 (130-200 μCi in 0.1 ml) was injected (i.v., tail vein) into 6 rats with CNS-1 brain tumor xenografts. MicroPET images (Concorde Microsystems-P4, 30 min acquisition) were acquired immediately after and at 6 h, 24 h, and 96 h post injection. Contrast-enhanced MRI images were obtained immediately following the final PET scan and PET/MRI images were manually fused using Amira (V3.1, TGS, Inc). Rats were euthanized and brain tissue subjected to histopathologic analysis.

Results

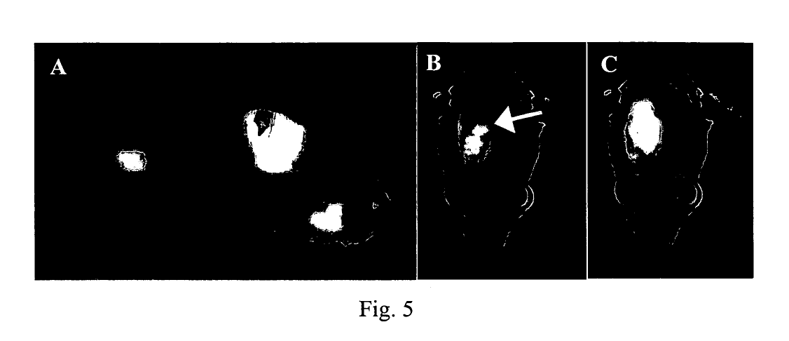

¹²⁴ I-NM404 showed no tumor uptake within 30 minutes of injection and respectable tumor uptake 6 hours following injection. Tumor conspicuity was significant, however, at either 24 or 96 hours. Little to no concomitant normal brain tissue activity was observed at any time point. Representative results obtained at 24 hours post-i.v. injection are shown in FIG. 5.

### EXAMPLE 4

### Evaluation of ¹²⁵I-NM404 in a Spontaneous Murine Pancreatic Adenocarcinoma Model

Materials and Methods

Mouse Pancreatic Adenocarcinoma Models: Two murine strains that are transgenic for either c-myc or k-ras, well-known oncogenes, have been developed at the University of Wisconsin (Sandgren E P, et al. Proc. Natl. Acad. Sci. USA 88:93-97, 1991 and Grippo P J, et al. Cancer Research 63:2016-2019, 2003). Expression of c-myc is targeted to pancreatic acinar cells because it is linked to an elastase promoter, which is only expressed in the pancreas. These ela-1-myc transgenic mice develop acinar and ductal neoplasia, which results in death between 2 and 7 months of age. By one month of age, the pancreas appears thickened and firm. Thus mice between the ages of 1-3 months serve as excellent models for the study of pancreatic cancer. Most human pancreatic neoplasms have a ductal morphology, and Dr. Sandgren's transgene targeting strategies are aimed at developing tumors that are specific for pancreatic ductal epithelium.

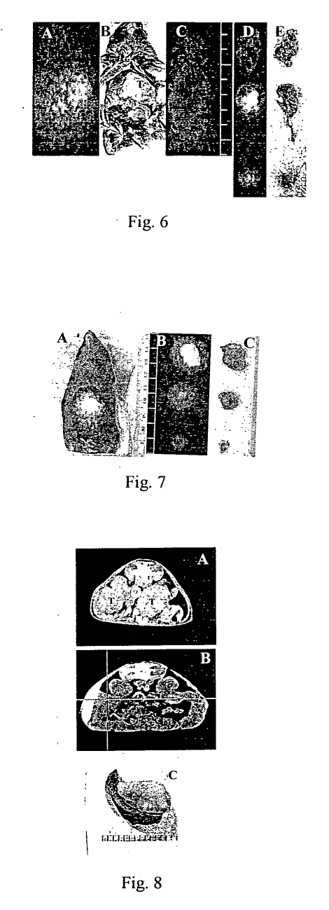

Imaging Studies: In order to determine if NM404 localizes in mouse pancreatic tumors, six c-myc transgenic mice were scanned on a Bioscan AR-2000 radioTLC scanner (modified by inventors for mouse imaging) from 2-21 days after tail vein injection of ²⁵¹I-NM404 (15 μCi/20 g body weight). On the last day, mice also underwent microCT scanning (42 kvp, 410 μA, 390 steps, MicroCAT-I, ImTek, Inc., Knoxville, Tenn.). Following in vivo imaging of anesthetized mice, the pancreatic tumors were excised and scanned ex vivo on the same scanner (equipped with high resolution 1 mm collimator and 2-D acquisition and analysis software) in order to avoid tissue attenuation associated with the low energy of iodine-125 (FIGS. 6 and 7). At sacrifice, tissues were excised, weighed, and radioactivity quantitated in a gamma counter.

Results

Imaging results with NM404 in the c-myc model indicated striking uptake and prolonged retention (>21 days) in all adenocarcinomas ranging from 5-12 mm in diameter.

NM404 is apparently metabolized and cleared from normal cells but becomes metabolically trapped in tumor cell membranes. Other autoradiography experiments in other tumor models showed that only viable tumor cells, and not normal tissue or necrotic tissues, are capable of accumulating NM404. We were also able to detect pancreatic tumors in live mice with microCT despite the ubiquitous nature of the pancreas in mice (FIG. 8).

### EXAMPLE 5 (PROPHETIC)

### Radioiodination of NM404 in Preparation for Clinical Use

A 2-ml glass vial is charged with 10 mg of ammonium sulfate dissolved in 50 μl of deionized water. Six 2 mm glass beads are added, then a Teflon-lined septum and screw cap are added and the vial gently swirled. A solution of 20 μg (in 20 μl of ethanol) of stock NM404 is added followed by aqueous sodium iodide (e.g., 125, 131, or 124, 1-5 mCi) in less than 30 μl aqueous 0.01 N sodium hydroxide. The isotope syringe is rinsed with three 20 μl portions of ethanol. The resulting reaction vial is swirled gently. A 5-ml disposable syringe containing glass wool in tandem with another 5-ml charcoal nugget filled syringe with needle outlet are attached. The glass wool syringe acts as a condensation chamber to catch evaporating solvents and the charcoal syringe traps free iodide/iodine. The resulting reaction vessel is heated in a heating block apparatus for 45 minutes at 150° C. Four 20 ml volumes of air are injected into the reaction vial with a 25-ml disposable syringe and allowed to vent through the dual trap attachment. The temperature is raised to 160° C. and the reaction vial heated another 30 minutes. After cooling to room temperature, ethanol (200 μl) is added and the vial swirled. The ethanolic solution is then passed through a pre-equilibrated Amberlite IRA 400 resin column to remove unreacted iodide. The eluent volume is reduced to 50 μl via a nitrogen stream (use charcoal syringe trap) and the remaining volume injected onto a silica gel column (Perkin Elmer, 3 μm×3 cm disposable cartridge column eluted at 1 ml/min with hexane/isopropanol/water (52:40:8)) for purification. Final purity is determined by TLC (plastic backed silica gel-60 eluted with chloroform-methanol-water (65:35:4, Rf=0.1). The HPLC solvents is removed by rotary evaporation and the resulting radioiodinated NM404 solubilized in aqueous 2% Tween-20 and passed through a 0.22 μm filter into a sterile vial.

### EXAMPLE 6 (PROPHETIC)

### ¹²⁴I-NM404-PET Imaging in Patients

¹²⁴I-NM404 maximum dose for human administration is calculated as follows: Animal biodistribution data is generated to determine the percentage of injected dose/organ at varying time points. These animal data are extrapolated to man by means of MIRD formalism (MIRDOSE PC v3.1) using standard conversion factors for differences in organ mass and anatomy between rat and standard man, providing predicted human organ doses. Based on these predicted doses, the permissible mCi dose to be injected into humans is determined using the maximal doses legally permitted by RDRC regulations for specific human tissue as defined in the Federal Register (21CFR Part 361.1). For example, based on the ¹³¹I-NM404 data it is expected that the maximum starting dosage for ¹²⁴I-NM404 should be below 2.0 mCi for pancreatic tumor imaging.

Patients receive SSKI (2 drops three times daily beginning 1 day before and continuing for seven days) in order to minimize uptake of free radioiodide by the thyroid. Patients allergic to iodine may be given potassium perchlorate (200 mg every 8 hours) starting one day before injection and continuing for 3 days post injection. ¹²⁴I-NM404 is administered intravenously over 5 minutes. A transmission scan using a Ga-68/Ge-68 rotating positron emitting pin source is performed to measure the attenuation. These data are used for attenuation correction of emission data.

The patients are scanned at one or more of the following multiple timepoints following infusion of the ¹²⁴I-NM-404: 90 minutes dynamic acquisition, 6 hours, 24 hours, 48 hours, and 96 hours.

The PET images are acquired in 2D mode with a BGO based GE ADVANCE PET scanner with an axial field of view of 152 mm. The images are acquired in 256×256 matrix and reconstruction is performed using a Hanning filter. All the images are attenuation corrected using the transmission data.

Before infusion, an intravenous line is established in the upper extremity. The ¹²⁴I-NM404 dose is measured in a dose calibrator prior to injection. A tracer dose of <2 mCi of ¹²⁴I-NM04 is infused over 2-5 minutes. The preparation is sterile, pyrogen-free, and contains <5% free iodine by thin layer chromatography (usual syntheses yield free radioiodine of ≦1%).

Phantom studies using ¹²⁴I is performed to determine the calibration factor for the PET scanner and well counter. Phantom studies are performed for the same imaging times and same duration of acquisition.

The influx constant of the target region of uptake for any given patient is compared to a background region in the same patient and the lesions is classified as tumor or non-tumor regions based on this comparison. Similar classification of tumor and non-tumor region can also be done by visual analysis.

The present invention is not intended to be limited to the foregoing examples, but encompasses all such modifications and variations as come within the scope of the appended claims.

We claim:

1. A method for detecting malignant breast or mammary tumor but not hyperplasia or benign tumor in a breast or mammary tissue of a human or non-human animal, the method comprising the steps of:
administering a phospholipid ether analog to the human or non-human animal wherein the phospholipid ether analog is selected from the compounds represented by the general formula I or II: 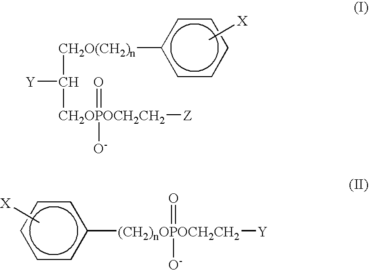
wherein in formula I X is a radioactive isotope of a halogen, n is an integer between 16 and 30, Y is selected from the group consisting of H, OH, COOH, O(C═O)R, and OR, and Z is selected from the group consisting of NH₂, NR₂, and NR₃, wherein R is an alkyl or aralkyl substituent, and
wherein in formula II X is a radioactive isotope of a halogen, n is an integer between 16 and 30, and Y is selected from the group comprising NH₂, NR₂, and NR₃, wherein R is an alkyl or aralkyl substituent; and
determining whether one breast or mammary region of the animal retains a higher level of the analog than surrounding breast or mammary regions wherein a higher retention region indicates the location of malignant breast or mammary tumor but not hyperplasia or benign tumor.

2. The method of claim 1, wherein X is a radioactive isotope of iodine in both formula I and II.

3. The method of claim 2, wherein X is I¹²⁴ in both formula I and II.

4. The method of claim 2, wherein the phopholipid ether analog is 18-(p-iodophenyl)-octadecylphosphocholine.

5. The method of claim 1, wherein the method is for differentiating between a benign and a malignant tumor in breast or mammary tissue.

6. A method for detecting brain tumor in a human or non-human animal that has or is suspected of having a brain tumor, the method comprising the steps of:
administering a phospholipid ether analog to the human or non-human animal wherein the phospholipid ether analog is selected from the compounds represented by the general formula I or II: 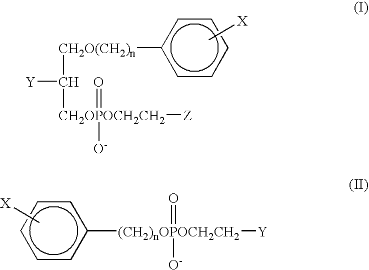
wherein in formula I X is a radioactive isotope of a halogen, n is an integer between 16 and 30, Y is selected from the group consisting of H, OH, COOH, O(C═O)R, and OR, and Z is selected from the group consisting of NH₂, NR₂, and NR₃, wherein R is an alkyl or aralkyl substituent, and
wherein in formula II X is a radioactive isotope of a halogen, n is an integer between 16 and 30, and Y is selected from the group comprising NH₂, NR₂, and NR₃, wherein R is an alkyl or aralkyl substituent; and
determining whether one brain region of the human or non-human animal retains a higher level of the analog than surrounding brain region wherein the presence of a higher retention region indicates that the human or non-human animal has brain tumor.

7. The method of claim 6, wherein the brain tumor is glioma.

8. The method of claim 6, wherein X is a radioactive isotope of iodine in both formula I and II.

9. The method of claim 8, wherein X is I¹²⁴ in both formula I and II.

10. The method of claim 8, wherein the phopholipid ether analog is 18-(p-iodophenyl)-octadecylphosphocholine.

11. A method for detecting pancreatic cancer in a human or non-human animal that has or is suspected of having pancreatic cancer, the method comprising the steps of:
administering a tumor type specific phospholipid ether analog to the human or non-human animal wherein the phospholipid ether analog is selected from compounds represented by the general formula I or II: 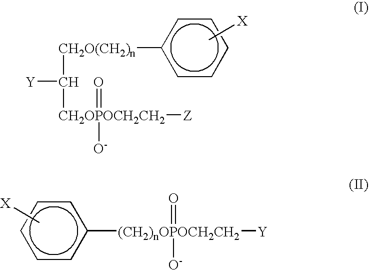
wherein in formula I X is a radioactive isotope of a halogen, n is an integer between 16 and 30, Y is selected from the group consisting of H, OH, COOH, O(C═O)R, and OR, and Z is selected from the group consisting of NH₂, NR₂, and NR₃, wherein R is an alkyl or aralkyl substituent, and
wherein in formula II X is a radioactive isotope of a halogen, n is an integer between 16 and 30, and Y is selected from the group comprising NH₂, NR₂, and NR₃, wherein R is an alkyl or aralkyl substituent; and
determining whether one pancreatic region of the human or non-human animal retains a higher level of the analog than surrounding pancreatic region wherein a higher retention region indicates that the human or non-human animal has pancreatic cancer.

12. The method of claim 11, wherein X is a radioactive isotope of iodine in both formula I and II.

13. The method of claim 12, wherein X is I¹²⁴ in both formula I and II.

14. The method of claim 12, wherein the phopholipid ether analog is 18-(p-iodophenyl)-octadecylphosphocholine.
